(19) United States
(12) Patent Application Publication
(10) Pub. No.: US 2005/0203287 A1
(43) Pub. Date: Sep. 15, 2005

(54) Process for the preparation of sulfamate derivatives

(76) Inventors: Chandrasekhar Batchu, Vashi (IN); Biju Kumar Gopinathen Pillai, Nerul (IN); Shekhar Bhaskar Bhirud, Vashi (IN)

Correspondence Address: DILWORTH & BARRESE, LLP, 333 EARLE OVINGTON BLVD., UNIONDALE, NY, 11553, US

(21) Appl. No.: 11/077,722

(22) Filed: Mar. 11, 2005

Related U.S. Application Data
(60) Provisional application No. 60/552,145, filed on Mar. 11, 2004.

(51) Int. Cl. C07D 317/44; C07H 5/06

(52) U.S. Cl. 536/18.7; 549/440; 558/47

(57) ABSTRACT

An improved process for the preparation of sulfamate derivatives such as topiramate is provided comprising (a) reacting an alcohol with sulfuryl chloride in the presence of an amine base and in a first halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof to produce a chlorosulfate intermediate and (b) reacting the chlorosulfate intermediate with an amine of the formula R¹NH₂, wherein R¹ is hydrogen or an alkyl from 1 to 4 carbon atoms, in a second halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof.

### CROSS-REFERENCE TO RELATED APPLICATIONS

This application claims the benefit under 35 U.S.C. §119 to Provisional Application No. 60/552,146, filed Mar. 11, 2004 and entitled “PROCESS FOR THE PREPARATION OF TOPIRMATE”, the contents of which are incorporated by reference herein.

### BACKGROUND OF THE INVENTION

1. Technical Field

The present invention generally relates to a process for the preparation of sulfamate derivatives.

2. Description of the Related Art

Generally, sulfamate derivatives of the general formula I: 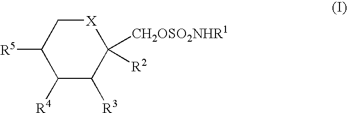
wherein X is CH₂ or O; and R¹, R², R³, R⁴ and R⁵ are as defined herein, are known compounds that have been found to exhibit anticonvulsant activity which may useful in the treatment of conditions such as epilepsy. See, e.g., U.S. Pat. Nos. 4,513,006; 4,582,916 and 5,387,700.

One such sulfamate derivative is topiramate (also known as 2,3:4,5-bis-O-(1-methylethylidene)-β-D-fructopyranose sulfamate) and is of the formula: 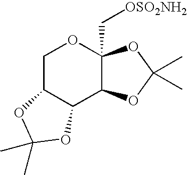
Topiramate exerts a potent anticonvulsant effect in maximal electroshock seizure (MES) assays. This compound is structurally distinct from known anticonvulsants in that it is a monosaccharide derivative and contains a sulfamate functional group. Anticonvulsants are the primary drugs used for the treatment of epileptic disorders. Topiramate is sold under the brand name Topamax®. See, e.g., The Merck Index, Thirteenth Edition, 2001, p. 1703, monograph 9625; and Physician's Desk Reference, “Topamax,” ₅₈^{th} Edition, pp. 2484-2490 (2003).

U.S. Pat. No. 4,513,006 to Maryanoff et al. (“the 006 patent”) and U.S. Pat. No. 4,582,916 to Maryanoff et al. (“the 916 patent”), herein incorporated by reference, disclose three different processes for preparing sulfamate derivatives such as topiramate. In the first process, an alcohol of the formula RCH₂OH is reacted with a chlorosulfamate of the formula ClSO₂NH₂ or ClSO₂NHR¹ in the presence of a base at a temperature in the range of −20° C. to 25° C. and in a solvent such as toluene, THF, or dimethylformamide wherein R is a moiety of formula II

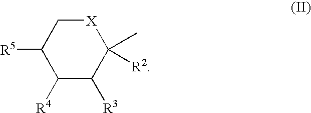

In the second process of the '006 and '916 patents, an alcohol of the formula RCH₂OH is reacted with sulfuryl chloride of the formula SO₂Cl₂ in the presence of a base such as triethylamine or pyridine at a temperature in the range of −40° C. to 25° C. and in a solvent such as diethyl ether or methylene chloride to produce a chlorosulfate of the formula RCH₂OSO₂Cl. The chlorosulfate may then be reacted with an amine of the formula R¹NH₂ at a temperature in the range of −40° C. to 25° C. and in a solvent such as methylene chloride or acetonitrile to produce the compound of the formula I.

In the third process, the chlorosulfate of the formula RCH₂OSO₂Cl is reacted with a metal azide in a solvent such as methylene chloride or acetonitrile to produce an azidosulfate of the formula RCH₂OSO₂N₃. The azidosulfate is then reduced to a compound of formula I wherein R¹ is hydrogen, by catalytic hydrogenation. The major drawbacks of these processes are (1) the first process uses a base and a solvent that are potentially explosive and requires a highly toxic and corrosive reagent to produce the starting materials; (2) the second process produces relatively low yields of the desired compound of formula I in comparison with the process of the present invention; and (3) the third process utilizes azides which may explode during handling. Also, another drawback with these processes is that the use of methylene chloride as a solvent is difficult to recover for recycling.

U.S. Pat. No. 5,387,700 to Maryanoff et al. (“the '700 patent”), herein incorporated by reference, discloses another process for the preparation of topiramate. The '700 patent uses a two step procedure. The first step of the process is the reaction of an alcohol of the formula RCH₂OH with sulfuryl chloride of the formula SO₂Cl₂ in presence of a tertiary or heterocyclic amine base in a solvent selected from toluene, t-butyl methyl ether or tetrahydrofuran to produce a chlorosulfate intermediate of the formula RCH₂OSO₂Cl. The second step is the reaction of the chlorosulfate intermediate with an amine of the formula R¹NH₂ in a solvent selected from t-butyl methyl ether, tetrahydrofuran or a lower alcohol to produce the compound of formula I. The major drawbacks of this process are that the ether solvents contain potentially explosive peroxides, tetrahydrofuran is problematic in recovery and recycling, the first step of the process contains numerous washing and isolation steps that are tedious and time consuming.

Accordingly, there remains a need for an improved process for preparing sulfamate derivatives such as topiramate that eliminates and reduces the drawbacks of the prior art in a convenient and cost efficient manner on a commercial scale which can be carried out under safe conditions and at relatively high purity levels.

### SUMMARY OF THE INVENTION

In one embodiment of the present invention, a process for preparing a compound of the formula I 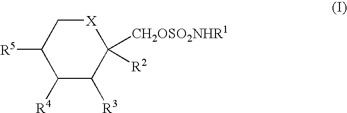
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 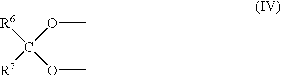
wherein R⁶ and R⁷ are the same or different and are hydrogen, alkyl or are alkyl joined together to form a cyclopentyl or cyclohexyl ring is provided comprising the steps of:
(a) reacting an alcohol of the formula RCH₂OH wherein R is a moiety of the formula II: 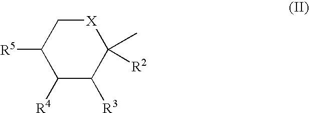
   wherein X, R², R³, R⁴ and R⁵ have the aforestated meanings with sulfuryl chloride in the presence of an amine base and in a first halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof to form a chlorosulfate intermediate of the formula III RCH₂OSO₂Cl; and
(b) reacting the chlorosulfate intermediate of formula III with an amine of the formula R¹NH₂ wherein R¹ has the aforestated meaning and in a second halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof to produce the compound of formula I.

Advantages of the present invention include at least:

1. The process is a one pot synthesis employing two steps that provides a simple and short route to the preparation of sulfamate derivatives.

2. The process may employ the same solvent for both steps of the synthesis, thereby reducing the load on solvent inventory and enabling better recoveries and recycling.

3. The second step of the process may be carried out by bubbling at room temperature and at atmospheric pressure. This alleviates the need for special equipment and safety precautions associated with pressure reactions.

4. The byproduct formed in the second step of the process, ammonium chloride, can be separated by conventional techniques, e.g., filtration, as it is relatively insoluble in the halogenated hydrocarbon solvents employed in the process of this invention

5. The process of this invention is much safer than the prior art processes which employ potentially explosive materials.

### DETAILED DESCRIPTION OF THE PREFERRED EMBODIMENTS

The present invention is directed to a process for synthesizing compounds of the following formula I: 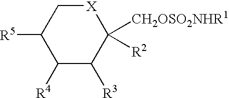
wherein X is CH₂ or O; R¹ is hydrogen or an alkyl from 1 to 4 carbon atoms, R², R³, R⁴ and R⁵ are independently hydrogen or alkyl of from 1 to 10 carbon atoms, and, when X is CH₂, R⁴ and R⁵ may be alkene groups joined to form a benzene ring and, when X is O, R² and R³ and/or R⁴ and R⁵, together, may be a methylenedioxy group of the following formula IV: 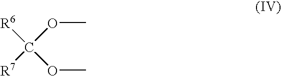
wherein R⁶ and R⁷ are the same or different and are hydrogen, alkyl or are alkyl joined together to form a cyclopentyl or cyclohexyl ring. In one embodiment, R⁶ and R⁷ may not both be hydrogen at the same time.

R¹ can be hydrogen or an alkyl of 1 to about 4 carbons, e.g., methyl, ethyl and isopropyl. The alkyl groups throughout this disclosure include straight and branched chain alkyl groups. Alkyl groups for R², R³, R⁴, R⁵, R⁶ and R⁷ are of 1 to about 10 carbon atoms and preferably from 1 to 3 carbon atoms. Examples of the alkyl groups include, but are not limited to, methyl, ethyl, isopropyl and n-propyl. When X is CH₂, R⁴ and R⁵ may combine to form a benzene ring fused to the 6-membered X-containing ring, i.e., R⁴ and R⁵ are defined by the alkatrienyl group ═CH—CH═CH—CH═.

This process is advantageously useful for producing compounds of formula I wherein X is oxygen and both R² and R³ and R⁴ and R⁵ together are methylenedioxy groups of the formula IV, wherein R⁶ and R⁷ are both hydrogen, both alkyl or combine to form a spiro cyclopentyl or cyclohexyl ring, in particular where R⁶ and R⁷ are both alkyl such as methyl.

The process of the present invention includes, in a first step, reacting a sufficient amount of an alcohol of the formula RCH₂OH wherein R is a moiety of Formula II: 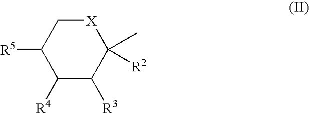
wherein X, R², R³, R⁴ and R⁵ have the aforestated meanings with sulfuryl chloride in the presence of an amine base at a temperature ranging from about −75° C. to about 50° C. and preferably from about 0° C. to about 40° C. and in a first halogenated hydrocarbon solvent selected from the group consisting of aliphatic halogenated hydrocarbon solvents with 1 to 12 carbon atoms and at least three halogen atoms, aliphatic halogenated hydrocarbon solvents with 2 to 12 carbon atoms and less than three halogen atoms and aromatic halogenated hydrocarbon solvents with 6 to 18 carbon atoms and mixtures thereof to produce a chlorosulfate intermediate of the formula III RCH₂OSO₂Cl wherein R has the aforestated meaning. The concentration of the alcohol can range from about 0.97 to about 1.0 molar equivalents per equivalent of the sulfuryl chloride. The time period for this reaction is generally from about 3 to about 4 hours.

The chlorosulfate intermediate of the formula III can then be stabilized by, for example, an aqueous wash from the product mixture, or by treatment with a base such as sodium bicarbonate, or preferably by aqueous wash and treatment with a base. The organic layer can then be separated from the reaction mixture by conventional techniques, e.g., filtration.

Next, the chlorosulfate intermediate of formula III is reacted with an amine of the formula R¹NH₂, wherein R¹ has the aforementioned meaning at a temperature ranging from about −50° C. to about 50° C. and preferably from about 0° C. to about 35° C. and in a second halogenated hydrocarbon solvent selected from the group consisting of aliphatic halogenated hydrocarbon solvents with 1 to 12 carbon atoms and at least three halogen atoms, aliphatic halogenated hydrocarbon solvents with 2 to 12 carbon atoms and less than three halogen atoms and aromatic halogenated hydrocarbon solvents with 6 to 18 carbon atoms and mixtures thereof to form the compounds of formula I, e.g., topiramate. The time period for this reaction is generally from about 8 to about 10 hours.

The reaction with the amine of the formula R¹NH₂ can be carried out using any appropriate amine source, e.g., ammonia gas (R¹═H) from an ammonia gas generating source such as aqueous or anhydrous ammonia under pressure of from about atmospheric to about 50 psi, and preferably from about 20 to about 30 psi. In another embodiment, the amine can be bubbled into the reaction solution.

Suitable amine bases for use in the first step of the present invention include, but are not limited to, primary amines, secondary amines, tertiary amines, aliphatic amines, aromatic amines and the like and mixtures thereof. In one embodiment, the amine base is a tertiary amine, heterocyclic amine and mixtures thereof. In another embodiment, the amine base is a trialkylamine, heterocyclic amine and mixtures thereof. Useful tertiary amine base groups include, but are not limited to, tri(lower alkyl)amines containing from about 4 to about 20 carbon atoms such as trimethylamine, triethylamine, tripropylamine, tributylamine and the like and mixtures thereof. Useful heterocyclic amine base groups include, but are not limited to, substituted and unsubstituted pyridines, substituted and unsubstituted morpholines, substituted and unsubstituted piperazines, substituted and unsubstituted piperidines, substituted and unsubstituted pyrrolidines, and the like and mixtures thereof. In another embodiment of the present invention, the amine base employed in the first step is selected from the group consisting of triethylamine, pyridine and pyridine derivatives. In a preferred embodiment of the present invention, the amine base is pyridine. Generally, the amine base is present in the range of about 1 to about 1.3 molar equivalents per equivalent of the alcohol.

The first halogenated hydrocarbon solvent is selected from the group consisting of aliphatic halogenated hydrocarbon solvents having 1 to 12 carbon atoms and at least three halogen atoms, e.g., chloroform, trichloroethylene, carbon tetrachloride, perchloroethylene and the like and mixtures thereof; aliphatic halogenated hydrocarbon solvents having 2 to 12 carbon atoms and less than three halogen atoms, e.g., ethylene dichloride, 1-chloropropane, 1,2-dichloropropane, isopropyl chloride, 1-chlorobutane, 1-chloropentane, 1-chlorooctane, 1-bromo-3-methylbutane, 1,2-dibromopropane, butyl bromide and the like and mixtures thereof; aromatic halogenated hydrocarbon solvents having 6 to 18 carbon atoms, e.g., chlorobenzene, dichlorobenzene and the like; and mixtures thereof. In one embodiment, the first halogenated hydrocarbon solvent is selected from the group consisting of ethylene dichloride, trichloroethylene, chloroform and mixtures thereof. In another embodiment, the first halogenated hydrocarbon solvent is ethylene dichloride. The ratio of the alcohol to solvent will ordinarily range from about 1:5 w/v to about 1:15 w/v, and preferably the ratio of alcohol to solvent is about 1:10 w/v.

The second halogenated hydrocarbon solvent can be the same as or different than the first halogenated hydrocarbon solvent and is selected from the group consisting of aliphatic halogenated hydrocarbon solvents having 1 to 12 carbon atoms and at least three halogen atoms, e.g., chloroform, trichloroethylene, carbon tetrachloride, perchloroethylene and the like and mixtures thereof; aliphatic halogenated hydrocarbon solvents having 2 to 12 carbon atoms and less than three halogen atoms, e.g., ethylene dichloride, 1-chloropropane, 1,2-dichloropropane, isopropyl chloride, 1-chlorobutane, 1-chloropentane, 1-chlorooctane, 1-bromo-3-methylbutane, 1,2-dibromopropane, butyl bromide and the like and mixtures thereof; aromatic halogenated hydrocarbon solvents having 6 to 18 carbon atoms, e.g., chlorobenzene, dichlorobenzene and the like; and mixtures thereof. In one embodiment, the second halogenated hydrocarbon solvent is selected from the group consisting of ethylene dichloride, trichloroethylene, chloroform and mixtures thereof. In another embodiment, the first and second halogenated hydrocarbon solvents are both ethylene dichloride. The ratio of the chlorosulfate intermediate of formula III to the second halogenated hydrocarbon solvent will ordinarily range from about 1:5 to about 1:50 w/v, and preferably from about 1:10 to about 1:15 w/v.

The starting materials of the alcohol of the formula RCH₂OH may be obtained commercially or can be synthesized by techniques well known in the art, see, e.g., U.S. Pat. Nos. 4,513,006 and 5,387,700, the contents of which are incorporated by reference herein. For example, a preferred alcohol for use herein 2,3:4,5-Bis-O-(1-methylethylidene)-β-D-fructopyranose can be obtained from fructose by adding concentrated sulfuric acid and then precipitating a solid out with an alkali metal hydroxide, e.g., sodium hydroxide. The precipitant is then washed with acetone. Alternatively, carboxylic acids and aldehydes of the formulae RCOOH and RCHO wherein R is as defined above may be reduced to compounds of the formula RCH₂OH by standard reduction techniques, e.g. reaction with lithium aluminum hydride, sodium borohydride or borane-THF complex in an inert solvent such as diglyme, THF or toluene at a temperature of about 0° C. to about 100° C.

In a preferred embodiment of the process of the present invention, topiramate is prepared by starting with fructose and adding concentrated sulfuric acid and then precipitating a solid out with sodium hydroxide. The precipitant is then washed with acetone. The resulting 2,3:4,5-Bis-O-(1-methylethylidene)-β-D-fructopyranose is reacted with sulfuryl chloride in the presence of pyridine in an ethylene dichloride solvent. The resulting 2,3:4,5-Bis-O-(1-methylethylidene)-β-D-fructopyranose sulfonyl chloride is then reacted with ammonia gas in the same ethylene dichloride solvent as generally set forth in Scheme I:

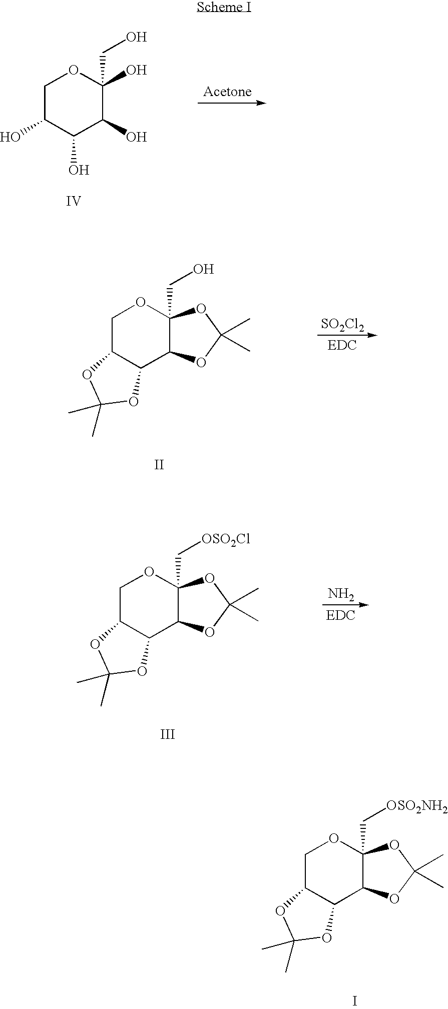

If desired, the product of the compounds (I) prepared by the process of the present invention can be purified may be recrystallized by conventional techniques, e.g., such as from ethanol/water or ethyl acetate/hexane. The process of the present invention advantageously provides compounds of formula I in relatively high purity, e.g., greater than about 97%, preferably greater than about 98% and more preferably greater than about 99%.

The following examples are provided to enable one skilled in the art to practice the invention and are merely illustrative of the invention. The examples should not be read as limiting the scope of the invention as defined in the claims.

### EXAMPLE 1

### Preparation of Starting Material: 2,3:4,5-Bis-O-(1-methylethylidene)-β-D-fructopyranose

Into a 20 L 4-necked round bottom flask, acetone (10 L) and fructose (1 kg) were added at a temperature of about 25° C. under stirring. The reaction mass was cooled to 0° C. and concentrated sulfuric acid (600 ml) was added to the reaction mixture. After completion of addition of concentrated sulfuric acid the reaction mixture was maintained at a temperature of about 25° C. under stirring for between 3 to 4 hours. The progress of the reaction was monitored by TLC. After completion of the reaction as determined by TLC, a 50% NaOH solution (4 L) was added in portions at a temperature of about 10° C. over about 30 minutes. The precipitated solids were filtered and the salt cake was washed with acetone (500 ml). The filtrate and washings were combined and then distilled under a vacuum below a temperature of about 65° C., until no more drops were observed. Isopropanol (300 ml) was added to the residue and the resulting isopropanol solution was added to n-hexane (2 L) under stirring. The resulting homogenous solution was added to water (1.5 L) and cooled to a temperature of about 10° C. under stirring. The precipitated solid was filtered and the solid was dispersed in a mixture of n-hexane (IL), isopropanol (100 ml), and water (500 ml) under stirring and filtered again. The operation of dispersing the solid in a mixture of n-hexane (IL), isopropanol (100 ml), and water (500 ml) was repeated. The resulting product was filtered and dried to below a temperature of about 50° C. until the moisture content reached below 1%. The dried product appears as an off-white crystalline solid. Net wt=about 1.3 kg, yield=83-90%, purity>98%, m.p. 87-92° C.

Step I—Preparation of Chlorosulfate Intermediate: 2,3:4,5-Bis-O-(1-methylethylidene)-β-D-fructopyranose sulfonyl chloride.

Into a 20 L 4-necked round bottom flask, ethylene dichloride (5 L) and sulfuryl chloride (700 g, about 422 ml) was added at a temperature of about 25° C. under stirring. The reaction mixture was cooled to a temperature of about −20° C. A solution of 2,3:4,5-Bis-O-(1-methylethylidene)-β-D-fructopyranose (1.0 kg) dissolved in ethylene dichloride (5 L) was added to the reaction mixture under stirring. Pyridine (365 ml) was added over about 3 hours at a temperature of about −20° C. After the addition, the reaction mixture was brought up to a temperature of about 25° C. and maintained at that temperature for between 3 to 4 hours. After completion of the reaction as determined by TLC, water (5 L) was added to the reaction mixture at a temperature of about 10° C. and stirred for about 15 minutes. The layers were separated and the organic layer was washed with sodium bicarbonate solution (3 L), followed by water (3 L). The organic layer was separated and filtered through a Hyflow bed, and then washed with ethylene dichloride (1 L). All the washings are then combined. The ethylene dichloride layer was concentrated in a rotavapor at a temperature of about 70° C. under reduced pressure until no more vapors were observed. The resulting residue appears as pale yellow oil. Net wt=about 1.3 kg, yield=94-98%, purity of about 97-98%.

Step II—Preparation of Topiramate.

Into the same 20 L 4-necked round bottom flask, ethylene dichloride (10 L) was added to the oily product of Step I containing 2,3:4,5-bis-O-(1-methylethylidene)-β-fructopyranose sulfonyl chloride (about 11 L). Charcoal (120 g) dispersed in ethylene dichloride (200 ml) was added at a temperature of about 25° C. under stirring. Dry ammonia gas was bubbled through the reaction mixture at a temperature of about 25° C. under stirring for between 10 to 12 hours. After completion of the reaction as determined by TLC, the mixture was filtered through a Hyflow bed and washed with ethylene dichloride (500 ml). All the washings were then collected. The ethylene dichloride layer was concentrated on a rotavapor at a temperature of about 65° C. under reduced pressure until no more drops were observed. The oil (about 1.25 kg) after distillation was dissolved in isopropanol (1.2 L) and ethyl acetate (0.6 L) and filtered through a Hyflow bed. The isopropanol and ethyl acetate solution was added to n-hexane (3.6 L) under stirring and maintained for about 1 hour at 25° C. The solution was cooled to a temperature of about 0° C. and maintained for about 1 hour under stirring. The precipitated solid was filtered and dried in a vacuum oven at a temperature of about 55° C. until the loss on drying reached below 1%. The product appears as an off-white to pale yellow solid. Net wt=about 975 g, yield=about 75%, purity of about 97-98%.

The product was dissolved in isopropanol (1.2 L) and ethyl acetate (0.6 L) and filtered through a Hyflow bed. The isopropanol and ethyl acetate solution was added to n-hexane (3.6 L) under stirring and maintained for about 1 hour at a temperature of about 25° C. The solution was cooled to a temperature of about 0° C. and maintained for about 1 hour under stirring. The dried solid appears as a white crystalline solid. Net wt=about 875 g, yield=about 67%, purity of >99.5%, mp 122-124° C., specific rotation [α]D 20=−33 (c=0.4% in MeOH).

IR (KBr) spectrum shows absorption bands 3385 cm⁻¹ (—N—H str), 3100 (—C—H str), 1390, 1186 cm⁻¹ (—SO2str). ¹H-NMR spectrum (CDCl₃, TMS as internal standard) shows δ 1.35, 1.40, 1.45, 1.55 (4S,12H,CH3), 3.8 (m,2H,H6), 4.1-4.4(m,4H), 4.5-4.7 (dd,1H4), 5.2 (br,2 NH₂). Mass: m/z 340 (M+).

### EXAMPLE 2

### Preparation of Starting Material: 2,3:4,5-Bis-O-(1-methylethylidene)-β-D-fructopyranose

Into a 5.0 L 4-necked round bottom flask, acetone (2.0 L) and D-fructose (200.0 g) were added at a temperature of about 25° C. under stirring. The reaction mass was cooled to −5 to 0° C. and concentrated sulfuric acid (120.0 ml) was added to the reaction mixture. After completion of addition of concentrated sulfuric acid the reaction mixture was maintained at a temperature of about 25-30° C. under stirring for between 3 to 4 hours. The progress of the reaction was monitored by TLC. After completion of the reaction as determined by TLC, the reaction mass was cooled to −5 to 0° C., and 50% NaOH solution (400 gm NaOH in 400 ml water) was added in portions at a temperature of about 10° C. over about 30 minutes. The precipitated solids were filtered and the salt cake was washed with acetone (400 ml). The filtrate and washings were combined and then distilled under a vacuum below a temperature of about 50° C., until no more drops were observed. Isopropanol (60 ml) was added to the residue and the resulting isopropanol solution was added to n-hexane (400 ml) under stirring. The resulting homogenous solution was added to water (300 ml) and the reaction mixture was heated to 50-55° C. for 30 minutes and cooled to a temperature of about 10-15° C. under stirring with stirring being continued for an additional one hour. The precipitated solid was filtered and washed a cake with a mixture of n-hexane (150 ml), isopropanol (20 ml), and water (150 ml). The wet cake was charged in 4N RBF with a mixture of n-hexane (500 ml), isopropanol (50 ml), and water (300 ml) and heated to 55-60° C. under stirring for 1 hour. The reaction mass was cooled to 5-10° C. under stirring with stirring continued for one hour at 5-10° C. The resulting product was filtered and the solids were washed with 100 ml chilled (10° C.) water and dried to a temperature below about 50° C. under vaccum until the moisture content reached below 1%.

Step 1—Preparation of Chlorosulfate Intermediate: 2,3:4,5-Bis-O-(1-methylethylidene)-β-D-fructopyranose sulfonyl chloride.

2,3:4,5-Bis-O-(1-methylethylidene)-β-D-fructopyranose obtained above was dissolved in six volume of ethylene dichloride under stirring. The reaction mass was cooled to −5 to −15° C. Pyridine (1.0 to about 1.3 mole equivalents with respect to starting material) was added to the reaction mass at −5 to −15° C. Into a 4-necked round bottom flask, a prepared sulfuryl chloride solution (four volumes with respect to starting material) in ethylene dichloride and added slowly to the reaction mixture of 2,3:4,5-Bis-O-(1-methylethylidene)-β-D-fructopyranose while maintaining the temperature between −5 to −15° C. After the completion of addition, the reaction mixture was brought up to a temperature of about 25° C. and maintained at that temperature for 2 hours. After completion of the reaction as determined by TLC, water (5 volumes with respect to starting material) was added to the reaction mixture at a temperature of about 10° C. and stirred for about 15 minutes. The layers were separated and the organic layer was washed with sodium bicarbonate solution (5 volumes with respect to starting material), followed by water (5 volumes with respect to starting material), followed by saturated brine solution (5 volumes with respect to starting material). The organic layer was separated and dried on anhydrous sodium sulfate, and then washed with ethylene dichloride. All the washings are then combined. The ethylene dichloride layer was concentrated under vaccum at a temperature of about 55° C.

Step II—Preparation of Topiramate.

Into the same 4-necked round bottom flask, ethylene dichloride (10 volumes with respect to 2,3:4,5-bis-O-(1-methylethylidene)-β-D-fructopyranose sulfonyl chloride) was added to the oily product of Step I containing 2,3:4,5-bis-O-(1-methylethylidene)-β-D-fructopyranose sulfonyl chloride. Dry ammonia gas was bubbled through the reaction mixture at a temperature of about 20-25° C. under stirring for between 10 to 12 hours. After completion of the reaction as determined by TLC, charcoal was added to the reaction mixture (10% w/w with respect to. starting material), stirred the reaction mixture for 1.0 hour, filtered through a Hyflow bed and washed with ethylene dichloride (2 volumes with respect to starting material). All the washings were then collected. The ethylene dichloride layer was concentrated on a rotavapor at a temperature below 50° C. under reduced pressure and degas for 30 minutes. The oil after distillation was dissolved in isopropanol (one volume with respect to starting material) and ethyl acetate (0.5 volume with respect to starting material) and heat the reaction mass to 50-55° C. to get the clear solution. To the isopropanol and ethyl acetate solution, n-hexane (4.5 volumes with respect to starting material) was added under stirring for about one hour and cooled the reaction mixture to 25-30° C. in 2 hours. The solution was cooled to a temperature of about 0-5° C. and maintained for about 2 hour under stirring. The precipitated solid was filtered and dried in a vacuum oven at a temperature of about 55° C. until the loss on drying reached below 1%. The product appeared as an off-white to pale yellow solid. Net wt=about 975 g, yield=about 75%, and purity of about 97-98%.

The product was dissolved in isopropanol and ethyl acetate and filtered through a Hyflow bed. The reaction mixture was heated to 50-55° C. to provide a clear solution. The n-hexane was added slowly to isopropanol and ethyl acetate solution under stirring and maintained for about 1 hour at a temperature of about 25° C. The solution was cooled to a temperature of about 0-5° C. and maintained for about 1 hour under stirring. The precipitated solid was filtered, washed with 0.5 volumes of n-hexane (with respect to crude topiramate) and then dried for 1.0 hour. Next, the dried product was dissolved in isopropanol and heated to 60 to 70° C. Water was added slowly to the isopropanol solution. The solution was cooled to a temperature of about 5-10° C. and maintained for about 2 hour under stirring. The precipitated solid was filtered and dried in a vacuum oven at a temperature of about 50-55° C. until the loss on drying reached below 1%.
Yield=about 67%, purity of >99.5%, mp 122-124° C., specific rotation [α]D 20=−33 (c=0.4% in MeOH).

IR (KBr) spectrum shows absorption bands 3385 cm⁻¹ (—N—H str), 3100 (—C—H str), 1390, 1186 cm⁻¹ (—SO2str). ¹H-NMR spectrum (CDCl₃, TMS as internal standard) shows δ 1.35, 1.40, 1.45, 1.55 (4S,12H,CH3), 3.8 (m,2H,H6), 4.1-4.4(m,4H), 4.5-4.7 (dd,1H4), 5.2 (br,2 NH₂). Mass: m/z 340 (M+).

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A process for preparing a compound of the formula I 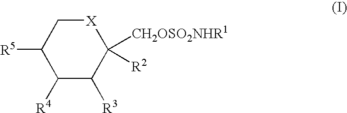
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 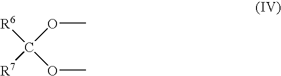
wherein R⁶ and R⁷ are the same or different and are hydrogen, alkyl or are alkyl joined together to form a cyclopentyl or cyclohexyl ring, the process comprising the steps of:
(a) reacting an alcohol of the formula RCH₂OH wherein R is a moiety of the formula II: 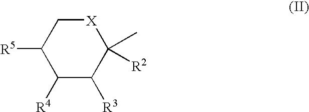
wherein X, R², R³, R⁴ and R⁵ have the aforestated meanings with sulfuryl chloride in the presence of an amine base and in a first halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof to form a chlorosulfate intermediate of formula III RCH₂OSO₂Cl; and,
(b) reacting the chlorosulfate intermediate of the formula III with an amine of the formula R¹NH₂ wherein R¹ has the aforestated meaning and in a second halogenated hydrocarbon solvent selected from the group consisting of aliphatic halogenated hydrocarbon solvents having 1 to 12 carbon atoms and at least three halogen atoms, aliphatic halogenated hydrocarbon solvents having 2 to 12 carbon atoms and less than three halogen atoms, aromatic halogenated hydrocarbon solvents having 6 to 18 carbon atoms and mixtures thereof to produce the compound of formula I.

2. The process of claim 1, wherein the alcohol is 2,3:4,5-Bis-O-(1-methylethylidene)-β-D-fructopyranose.

3. The process of claim 1, wherein the amine base is selected from the group consisting of trialkyl amines containing from about 4 to about 20 carbon atoms, heterocyclic amines and mixtures thereof.

4. The process of claim 1, wherein the amine base is selected from the group consisting of triethylamine, substituted and unsubstituted pyridines and pyridine derivatives thereof.

5. The process of claim 1, wherein the first and second halogenated hydrocarbon solvents are the same solvent.

6. The process of claim 1, wherein the first halogenated hydrocarbon solvent is selected from the group consisting of ethylene dichloride, trichloroethylene, chloroform and mixtures thereof and the second halogenated hydrocarbon solvent is selected from the group consisting of ethylene dichloride, trichloroethylene, chloroform and mixtures thereof.

7. The process of claim 1, wherein the first and second halogenated hydrocarbon solvents are ethylene dichloride.

8. The process of claim 1, wherein the ratio of the alcohol to the first halogenated hydrocarbon solvent is from about 1:5 w/v to about 1:15 w/v.

9. The process of claim 1, wherein the ratio of the alcohol to the first halogenated hydrocarbon solvent to alcohol is about 1:10 w/v.

10. The process of claim 1, wherein the ratio of the chlorosulfate intermediate of formula III to the second halogenated hydrocarbon solvent is about 1:5 to about 1:50 w/v.

11. The process of claim 1, wherein the ratio of the chlorosulfate intermediate of formula III to the second halogenated hydrocarbon solvent is about 1:10 to about 1:15 w/v.

12. The process of claim 1 wherein the chlorosulfate intermediate of formula III is 2,3:4,5-bis-O-(1-methylethylidene)-β-D-fructopyranose sulfonyl chloride.

13. The process of claim 1, wherein the chlorosulfate intermediate of formula III is stabilized by an aqueous wash and/or treatment with a base prior to its reacting in step (b).

14. The process of claim 1, wherein the amine of the formula R¹NH₂ is ammonia.

15. The process of claim 1, wherein in step (b) the reaction of the chlorosulfate intermediate of formula III with the amine of the formula R¹NH₂ is carried out by bubbling the amine into a solution containing the compound of formula III.

16. The process of claim 1, further comprising the step of recrystallizing the compound of formula I.

17. The process of claim 16, wherein the recrystallization step is carried out using a recrystallization medium selected from an alcohol and water, or ethyl acetate and hexane.

18. The process of claim 1, wherein the compound of formula I is

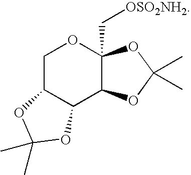

19. A process for preparing a compound of the formula I 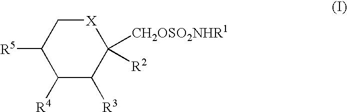
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 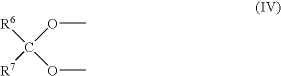
wherein R⁶ and R⁷ are the same or different and are hydrogen, alkyl or are alkyl joined together to form a cyclopentyl or cyclohexyl ring, the process comprising the steps of:
(a) reacting an alcohol of the formula RCH₂OH wherein R is a moiety of the formula II: 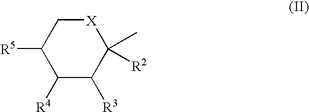
wherein X, R², R³, R⁴ and R⁵ have the aforestated meanings with sulfuryl chloride in the presence of an amine base selected from the group of triethylamine, substituted and unsubstituted pyridines and pyridine derivatives thereof and in an ethylene dichloride solvent to form a chlorosulfate intermediate of the formula III RCH₂OSO₂Cl; and,
(b) reacting the chlorosulfate intermediate of the formula III with an amine of the formula R¹NH₂ wherein R¹ has the aforestated meaning and in an ethylene dichloride solvent to produce the compound of formula I.

20. The compound of the formula I 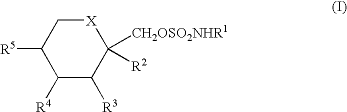
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 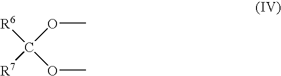
wherein R⁶ and R⁷ are the same or different and are hydrogen, alkyl or are alkyl joined together to form a cyclopentyl or cyclohexyl ring; prepared in accordance with the process of claim 1 having a purity greater than about 97%.

21. The compound of the formula I 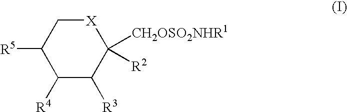
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 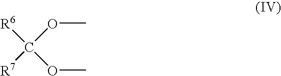
wherein R⁶ and R⁷ are the same or different and are hydrogen, alkyl or are alkyl joined together to form a cyclopentyl or cyclohexyl ring; prepared in accordance with the process of claim 1 having a purity greater than about 99%.

22. A compound of the formula I 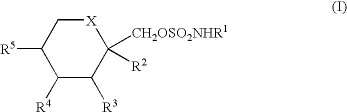
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 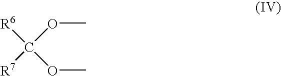
wherein R⁶ and R⁷ are the same or different and are hydrogen, alkyl or are alkyl joined together to form a cyclopentyl or cyclohexyl ring; having a purity greater than about 97%.

23. A compound of the formula I 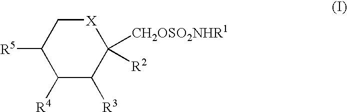
wherein X is CH₂ or O; R¹ is hydrogen or an alkyl from 1 to 4 carbon atoms; R², R³, R⁴ and R⁵ are independently hydrogen or alkyl, and, when X is O, R² and R³ and/or R⁴ and R⁵, together, may be a methylenedioxy group of the following formula IV: 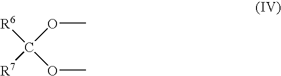
wherein R⁶ and R⁷ are the same or different and are hydrogen, alkyl or are alkyl joined together to form a cyclopentyl or cyclohexyl ring, having a purity greater than about 99%.
